(19) United States
(12) Patent Application Publication
(10) Pub. No.: US 2008/0167686 A1
(43) Pub. Date: Jul. 10, 2008

(54) Non-Rigid Intervertebral Spacers

(75) Inventors: Hai H. Trieu, Cordova, TN (US); Dimitri K. Protopsaltis, Memphis, TN (US)

Correspondence Address: HAYNES AND BOONE, LLP, 901 Main Street, Suite 3100, Dallas, TX, 75202, US

(73) Assignee: WARSAW ORTHOPEDIC, INC., Warsaw, IN (US)

(21) Appl. No.: 11/620,364

(22) Filed: Jan. 5, 2007

(51) Int. Cl. A61F 2/44; A61B 17/70

(52) U.S. Cl. 606/249; 623/17.16; 606/279

(57) ABSTRACT

An intervertebral spacer includes a non-rigid body having an upper beam member and a lower beam member. The upper beam member may include a lower inner surface and may include an upper outer surface configured to interface with a vertebral plate of an upper vertebra. The lower beam member may include an upper inner surface and may include a lower outer surface configured to interface with a vertebral plate of a lower vertebra. The upper inner surface of the lower beam member and the lower inner surface of the upper beam member may define an oval-shaped hollow portion.

### FIELD OF THE INVENTION

This disclosure is generally directed to prostheses and methods of implanting the prostheses, and more particularly, to intervertebral spacers and methods of implanting the intervertebral spacers in intervertebral spaces.

### BACKGROUND

Spinal discs between the endplates of adjacent vertebrae in a spinal column of the human body provide critical support. However, due to injury, degradation, disease or the like, these discs can rupture, degenerate and/or protrude to such a degree that the intervertebral space between adjacent vertebrae collapses as the disc loses at least a part of its support function. This can cause impingement of the nerve roots and severe pain. In some cases, surgical correction may be required.

Some surgical corrections include the removal of the natural spinal disc from between the adjacent vertebrae. In order to preserve the intervertebral disc space for proper spinal-column function, a rigid spacer can be inserted between the adjacent vertebrae.

Typically, conventional spinal spacers are implanted anteriorly between the adjacent vertebrae. Because anterior procedures often require displacement of organs, such as the aorta and vena cava, they must be performed with great care. Further, because scar tissue may grow about the surgical site, any required second treatment can be more difficult, and may introduce additional distress to the patient.

What is needed is an intervertebral spacer that is simple and allows posterior implantation. The intervertebral spacers disclosed herein address one or more deficiencies in the art.

### SUMMARY

In one exemplary aspect, this disclosure is directed to an intervertebral spacer including a non-rigid body having an upper beam member and a lower beam member. The upper beam member may include a lower inner surface and may include an upper outer surface configured to interface with a vertebral plate of an upper vertebra. The lower beam member may include an upper inner surface and may include a lower outer surface configured to interface with a vertebral plate of a lower vertebra. The upper inner surface of the lower beam member and the lower inner surface of the upper beam member may define an oval-shaped hollow portion.

In another exemplary aspect, this disclosure is directed to an intervertebral spacer including a non-rigid body having an upper beam member and a lower beam member. The upper beam member may include an arcing upper outer surface configured to interface with a vertebral plate of an upper vertebra. The lower beam member may include an arcing lower outer surface configured to interface with a vertebral plate of a lower vertebra. The body also may include a hollow portion between the upper and lower outer surfaces. The upper and lower beam members may connect in a manner such that the body can be compressed from a first height to a second smaller height.

In another exemplary aspect, this disclosure is directed to a method of surgically implanting an intervertebral spacer. The method may include accessing an intervertebral space defined by an upper vertebra and a lower vertebra. A non-rigid intervertebral spacer having a body with an upper beam member and a lower beam member may be introduced into the intervertebral space so that an arcing upper outer surface of the upper beam member interfaces with a vertebral plate of the upper vertebra, and so that an arcing lower outer surface of the lower beam member interfaces with a vertebral plate of the lower vertebra. The body may be compressed from a first height to a second smaller height.

### BRIEF DESCRIPTION OF THE DRAWINGS

FIG. 1 is an illustration of a side elevation view of an adult human vertebral column.

FIG. 2 is an illustration of a side view of a portion of the column of FIG. 1, depicting an exemplary intervertebral spacer between two adjacent vertebrae.

FIG. 3 is an illustration of an isometric view of an exemplary intervertebral spacer.

FIGS. 4A and 4B are illustrations of side views of the exemplary intervertebral spacer of FIG. 3 in compressed and uncompressed conditions.

FIGS. 5-11 are illustrations of additional exemplary implantable intervertebral spacers.

FIGS. 12 and 13 are illustrations of additional exemplary implantable intervertebral spacers having connecting elements.

FIGS. 14A and 14B show the intervertebral spacer of FIG. 13 in compressed and uncompressed conditions.

### DETAILED DESCRIPTION

This disclosure relates generally to an implantable non-rigid intervertebral spacer. For the purposes of promoting an understanding of the principles of the intervertebral spacer, reference will now be made to embodiments or examples illustrated in the drawings and specific language will be used to describe the same. It will nevertheless be understood that no limitation of the scope of the invention is thereby intended. Any alterations and further modifications of the described embodiments and any further applications of the principles of the invention as described herein are contemplated as would normally occur to one skilled in the art to which this disclosure relates.

The non-rigid intervertebral spacers disclosed herein may be implanted to maintain a height of a vertebral space and support adjacent vertebral bodies while allowing spinal motion. Compressing or flexing the spacers from a greater height to a lower height during implantation may minimize the required size of the surgical access site. Once implanted, the spacer height may elastically or mechanically increase to maintain the vertebral space and support the vertebrae. In the vertebral space, the spacer may provide, in some embodiments, axial compression and shock absorption via deformation under load. Its shape may allow spinal flexion motion and extension motion through rocking motion, while also allowing some lateral bending motion by compressing the spacer on a bending side. Its shape also may match the concave curvature of the adjacent vertebral plates and in some embodiments, the vertebral plates slide and rock over the spacer.

FIG. 1 illustrates a lateral view of a portion of a spinal column 10, illustrating a group of adjacent upper and lower vertebrae V1, V2, V3, V4 separated by natural intervertebral discs D1, D2, D3. Although the illustration generally depicts the lumbar region, it is understood that the devices, systems, and methods of this disclosure also may be applied to all regions of the vertebral column, including the cervical and thoracic regions.

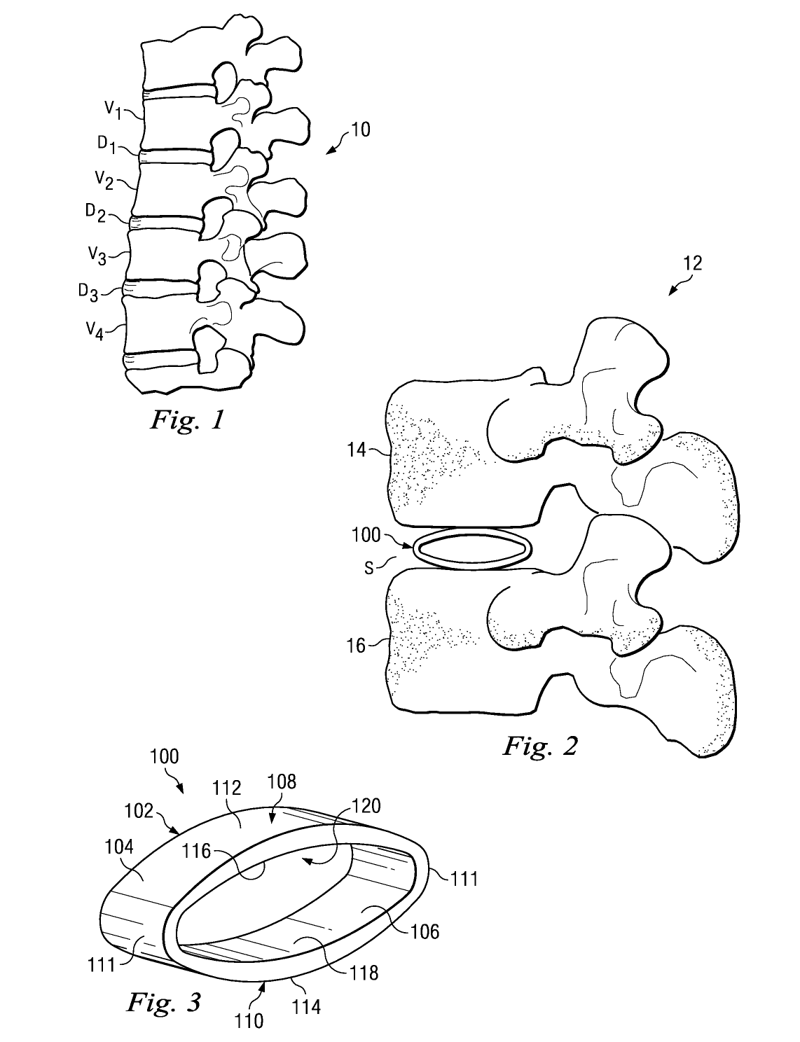

A joint comprises two adjacent vertebrae separated by an intervertebral disc. FIG. 2 illustrates an exemplary vertebral joint 12 including an upper vertebra 14 and a lower vertebra 16. In this illustration, instead of a natural intervertebral disc, a non-rigid vertebral spacer 100 is disposed between the upper and lower vertebrae 14, 16 and in contact with the vertebral endplates of the vertebral bodies. Sized to fit the disc space height in a manner similar to a natural intervertebral disc, such as any of discs D1-D4 in FIG. 1, the spacer 100 provides support and stabilization to the vertebrae. In addition, the spacer 100 also allows the upper vertebra 14 to move relative to the lower vertebra 16 to provide some movement to the joint. In the embodiment shown, the spacer 100 is a prosthetic device capable of compressing and flexing from a first height to a second height and back again. It also may tilt or change pitch in either the sagittal, axial, or coronal planes.

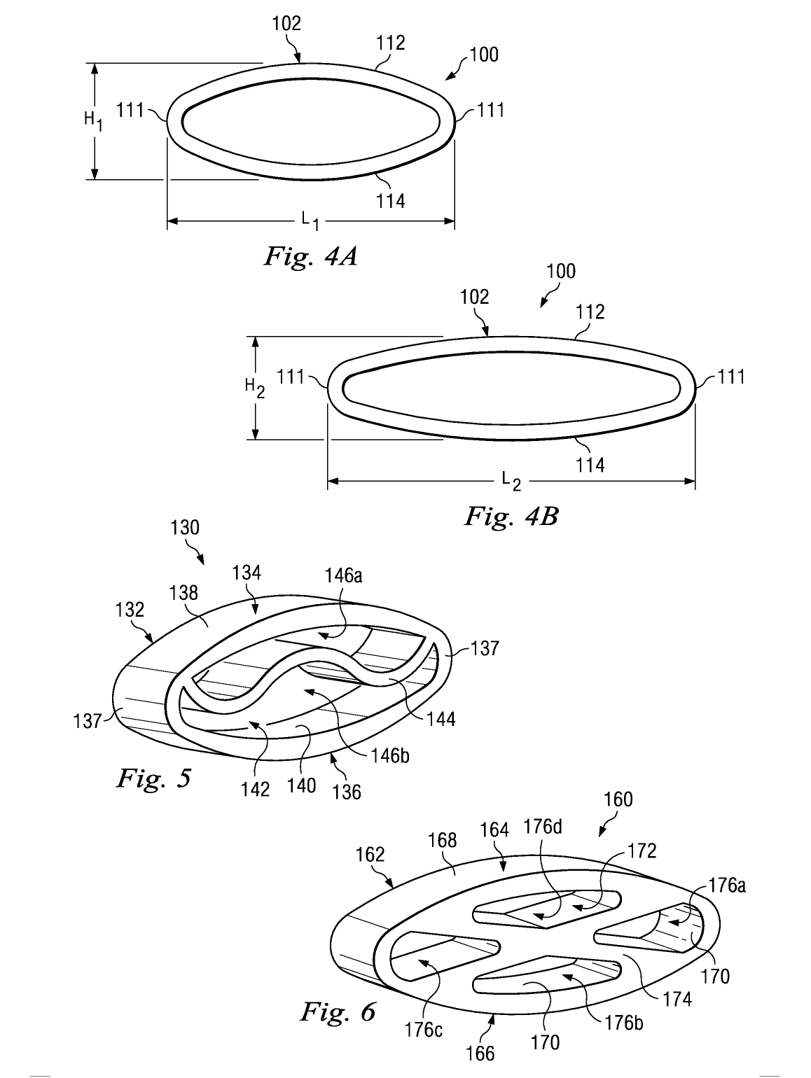

The spacer 100 is illustrated in greater detail in FIGS. 3, 4A, and 4B. FIG. 3 shows an isometric view and FIGS. 4A and 4B show side views of the spacer 100 in an uncompressed condition and a compressed condition respectively. Referring to FIG. 3, the spacer 100 includes a body 102 having an exterior surface 104 and an inner sidewall 106. In the embodiments shown, integral arcing upper and lower beam members 108, 110 connected at ends 111 form the body 102, giving it an elliptical or oval shape. Here, the upper and lower beam members are symmetrically disposed above and below a longitudinal centerline 113. The upper beam member 108 includes an upper outer surface 112 and the lower beam member 110 includes a lower outer surface 114. These outer surfaces 112, 114 form at least a part of the exterior surface 104. In addition, the upper beam member 108 includes an upper inner surface 116 and the lower beam member 110 includes a lower inner surface 118. These inner surfaces 116, 118 form at least a part of the inner sidewall 106. The inner sidewall 106 defines a hollow portion 120.

In FIG. 3, the outer surfaces 112, 114 of the beam members 108, 110 have an arcing convex shape. In some embodiments, this shape generally matches the concave shape of the vertebral endplates of the adjacent vertebrae. Accordingly, the matching shapes may assist in maintaining the spacer 100 within the intervertebral space between the vertebrae. In some embodiments, the outer surfaces 112, 114 act as articulating surfaces with the vertebral endplates by slidably supporting the vertebral endplates while providing a rocking motion. In addition, the arcing convex shape of the beam members 108, 110 makes the spacer 100 compressible and resilient. Thus, axial compression of the spacer 100 and shock absorption occurs via deformation of the device.

The spacer 100 may be formed of any suitable biocompatible material, including, for example, metals such as cobalt-chromium alloys, titanium alloys, nickel titanium alloys, and/or stainless steel alloys. Some embodiments of the spacer 100 are formed of any member of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); and/or cross-linked UHMWPE, among others. In the embodiment shown, the spacer 100 is integrally formed of a single material. Yet in other embodiments, multiple materials may be used. For example, the upper beam member 108 may be formed of a first material and the lower beam member 110 may be formed of a second different material. In such embodiments, some elements of the spacer 100 may be formed of a non-rigid material while other elements of the spacer 100 are formed of a rigid material, such as a rigid metal.

The outer surfaces 112, 114 may include features or coatings which enhance the fixation of the spacer 100 to the vertebral endplates of the vertebrae 14, 16. For example, the surfaces 112, 114 may be roughened such as by chemical etching, bead-blasting, sanding, grinding, serrating, and/or diamond-cutting. All or a portion of the outer surfaces 112, 114 may also be coated with a biocompatible and osteoconductive material such as hydroxyapatite (HA), tricalcium phosphate (TCP), and/or calcium carbonate to promote bone in growth and fixation. Alternatively, osteoinductive coatings, such as proteins from transforming growth factor (TGF) beta superfamily, or bone-morphogenic proteins, such as BMP2 or BMP7, may be used. Other suitable features may include spikes, ridges, and/or other surface textures and features.

Under normal spinal loads applied at the upper and lower outer surfaces 112, 114, the body 102 may appear as shown in FIG. 4A, having a first height H1 and a first length L1. The body 102 may be designed and formed to maintain a selected height of the intervertebral disc space and to support the adjacent vertebral bodies. However, because the non-rigid spacer 100 is compressible and resilient, the non-rigid body 102 may deform under applied axial forces to appear as shown in FIG. 4B, having a second height H2 and a second length L2. The second height H2 is less than the first height H1 and the second length L2 is greater than the first length L1. The ability of the spacer 100 to deformably compress from the first height H1 to the second height H2 may provide shock absorption properties when spinal loads exceed normal spinal loads, as may occur, for example, during active physical activities, such as jumping.

In addition, the flexibility of the spacer 100 may provide support during spinal flexion and extension, where the direction of applied loads may further depart from straight axial loads. This flexibility may allow the spinal flexion and extension motion. Also, the flexibility of the spacer 100 may allow less invasive implantation techniques to be used. For example, the spacer 100 may be introduced posteriorally or laterally in the lower second height H2, and then once in the vertebral space, expanded or released to elastically or mechanically return to its greater first height H1.

The spacer 100 also includes a width W1 (shown in FIG. 3) that is less than the length L1 (shown in FIG. 4a) when the spacer is in the uncompressed condition. In the embodiment shown, the length L1 is more than 20% greater than the width W1. In some embodiments, the length L1 is more than 50% greater than the width W1, and in others, more than double the width W1. Such a width to length ratio allows the spacer 100 to be introduced into a disc space using minimally invasive procedures, with a minimum incision size and access window. This reduces patient trauma and can speed recovery.

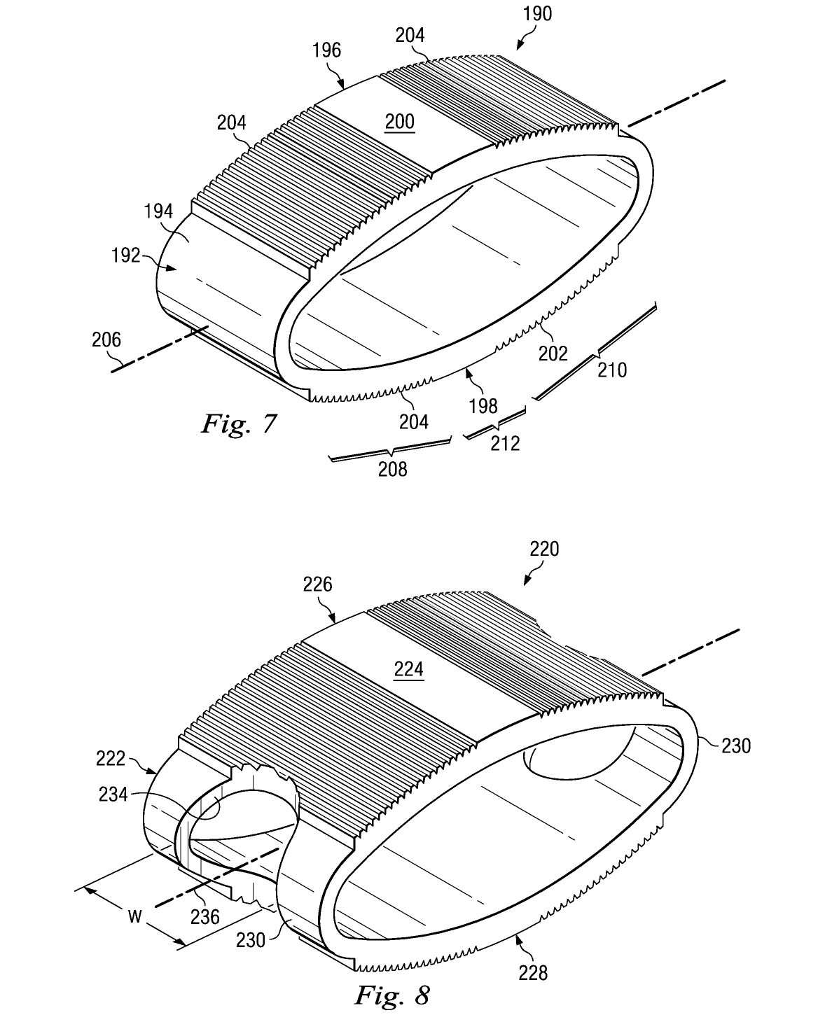

FIGS. 5 and 6 show alternative embodiments of exemplary spacers. FIG. 5 shows a spacer 130 having some features similar to those described in other embodiments herein, including a body 132 formed of upper and lower beam members 134, 136 that connect at ends 137 and that has an exterior surface 138 and has an inner side wall 140 defining a hollow portion 142. In this embodiment, the spacer 130 also includes a support member 144 that extends across at least a portion of the hollow portion from one portion of the inner sidewall 140 to another portion of the inner sidewall 140, dividing the hollow portion 142 into first and second regions 146a, 146b. In this embodiment, the support member 144 connects to and extends from end regions, near the ends 137 of the beam members 134, 136. In the embodiment shown, the support member 144 connects to the upper beam member in the end regions, but in other embodiments, the support member could connect to the lower beam member 136, could connect to both the upper and lower beam members 134, 136, and could connect to the ends 137, among other locations.

FIG. 6 shows a spacer 160 having some features that may be similar to those described above, including a body 162 formed of upper and lower beam members 164, 166 and having an exterior surface 168 and an inner side wall 170 defining a hollow portion 172. In this embodiment, the spacer 160 also includes a support member 174 that extends from one portion of the inner sidewall 170 to another portion of the inner sidewall 170 within the hollow portion 172. In this embodiment, the support member 174 attaches to the side wall 170 at four locations and the support member 174 forms an x-shape within the hollow portion 172, dividing the hollow portion 172 into four regions 176a-d.

The support members 144, 174 affect the flexibility and compressive properties of the respective spacers 130, 160. Their shape may provide symmetric or non-symmetric compressive characteristics. These compressive characteristics may at least partially determine how the spacers respond under load, in flexion and extension, and in lateral bending. It should be noted that although the support member 144 is a wavy or sinusoidal shape and the support member 174 is an X-shape, support members may be in any shape that provides support to the spacer under load and affects its compressive characteristics. For example, other support members may be shaped in a straight line, as a Y-shape, or other shape. In some embodiments, the spacers include more than one support member within the hollow portions, independent from each other. For example, the spacers may include two support members that form a V-shape or that don't contact to each other at all. Other shapes also are contemplated.

FIG. 7 illustrates another exemplary aspect of a spacer in accordance with the present disclosure. The spacer 190 may include some features that may be similar to other embodiments described herein, including a body 192 having an exterior surface 194 and being formed of upper and lower beam members 196, 198. The upper and lower beam members 196, 198 include respective arcing or curved upper and lower outer surfaces 200, 202. In this embodiment, bone engaging features 204 are formed on the upper and lower outer surfaces 200, 202. These features are configured to interface with concave vertebral endplates and increase frictional characteristics at the interface. In this embodiment, the bone engaging features 204 are formed as a plurality of parallel ridges extending substantially transverse to a longitudinal axis 206 of the body 192. Formed on only a portion of the upper and lower outer surfaces 200, 202, these ridges engage with and reduce sliding of the spacer 190 relative to the adjacent vertebral bodies. In the embodiment shown, the bone engaging features 204 are formed on a first and a second region 208, 210 of the upper and lower beam members 196, 198, with each region separated by a relatively smoother region 212. These bone engaging features 204 may assist in limiting sliding motion between the outer surfaces 200, 202 and the adjacent vertebrae, finding particular usefulness when the spacer is intended for fusion procedures.

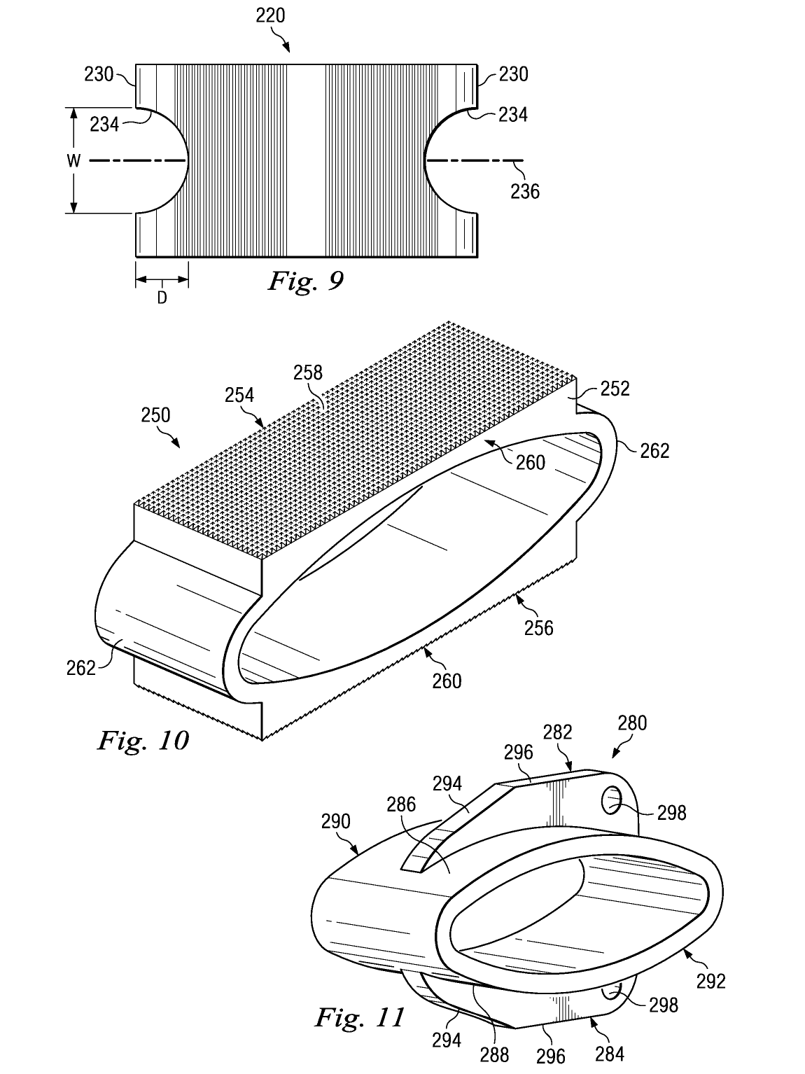

FIGS. 8 and 9 illustrate another exemplary aspect of a spacer 220 in accordance with the present disclosure including a body 222 having an exterior surface 224 and having beam members 226, 228 connected at ends 230. This embodiment includes some features similar to those of FIG. 7, but also includes a tension adjustment cutout 234. In the embodiment shown, the cutout 234 is a semicircular-shaped portion symmetrically located about a longitudinal centerline 236. The cutout 234 extends through the ends 230 thereby being formed in both the upper and lower beam members 226, 228. Adjusting a width W2 and a depth D of the cutout 234 affects the spring rate of the spacer 220, thereby changing its rigidity, flexibility, and dampening properties.

In some embodiments, the cutout 234 is symmetrically centered about the longitudinal centerline 236, while in others it is offset toward one side of the centerline 236. In these embodiments, the rigidity or flexibility of the spacer is offset, providing more support along one side of the spacer than the other. Additional embodiments include a first cut-out, such as at one end, sized differently than a second cut-out, such as at the other end or alternatively, a cutout at one end without a cutout at the other end. Other embodiments include multiple cutouts located at the ends or in some alternative embodiments, along the beam members. In yet other embodiments, the cutouts are formed not at the ends 230, but are formed elsewhere in the beam members 226, 228. Other variations also are contemplated.

It should be noted that the spacer's flexibility and rigidity also may be controlled using the structure of the body. For example, some regions of the body, such as the ends, may be formed to have a cross-sectional thickness different than at other regions, such as the central areas of the beam members. Other embodiments have a greater cross-sectional thickness at the beam members than at the ends. Still other arrangements are contemplated. The varying thickness can be used to provide desired rigidity characteristics, such as flexibility and spring rate.

FIG. 10 is an illustration of another spacer 250 according to another exemplary aspect of this disclosure. This embodiment includes some features similar to those described above, but also includes reinforced blocking 252 formed on upper and lower beam members 254, 256. The reinforced blocking 252 provides an interfacing surface 258 having a radius of curvature different than that of prior embodiments. In this embodiment, the reinforced blocking 252 provides a substantially flat surface for interfacing with the vertebral endplates. In addition, the reinforced blocking 252 changes the thickness of the beam members, with the beam members 254, 256 being least thick at center regions 260 and being progressively thicker as the distance from the center regions increases. Spacer ends 262 are un-reinforced, providing a desired rigidity and flexibility thereby allowing the spacer to deform and dampen under loads.

In this embodiment, the flat interfacing surface 258 includes bone engaging features 264 formed thereon. Here the bone engaging features 264 include a knurled surface formed of multiple protuberances. In other embodiments, spikes, protrusions, angled ridges, or other surface features make up the bone engaging features.

FIG. 11 illustrates yet another exemplary embodiment. This embodiment of the spacer 280 may formed similar to those described above, but includes upper and lower keels 282, 284 extending outwardly from upper and lower outer surfaces 286, 288 of upper and lower beam members 290, 292. In the exemplary embodiment shown, the keels 282, 284 each include a tapering leading edge 294 followed by a more level surface 296. This tapered leading edge 294 may ease the introduction process. The keels 282, 284 may include insertion tool connecting features 298 configured to interface with an insertion tool (not shown). Here, the insertion tool connecting features 298 are holes through the keels 282, 284 sized to connect with the insertion tool to hold the spacer 280.

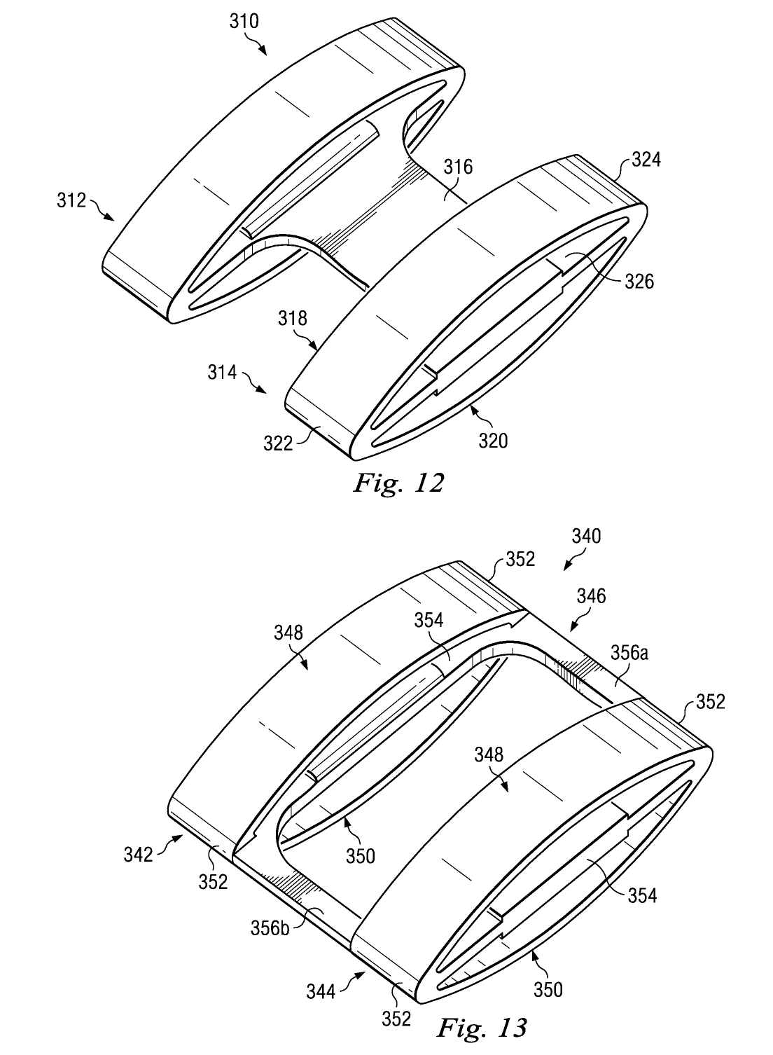

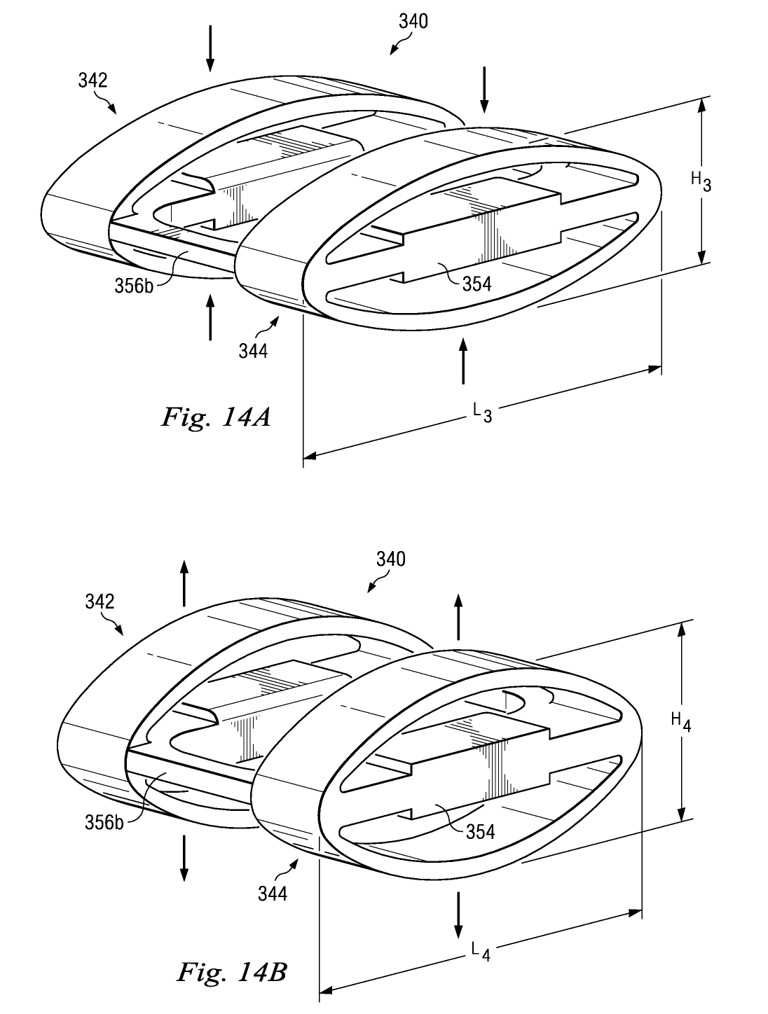

FIGS. 12 and 13 show additional alternative embodiments of non-rigid spacers. These spacers include a plurality of bodies connected by at least one stabilizing connecting element. FIG. 12 shows a spacer 310 formed of bodies 312, 314 and a connecting element 316. The bodies 312, 314 may include any features of the bodies described herein. Because in the embodiment shown, the bodies 312, 314 are substantially the same, only the body 314 is described here. Nevertheless, it is understood that the bodies may be formed to have features or rigidity characteristics that vary from one body to the other. The body 314 includes upper and lower beam members 318, 320 connected at ends 322, 324. A support member 326 extends from one end to the other end.

In some embodiments, the support member 326 may be as described above, while in other embodiments the support member 326 is an extending and retracting actuator that operates to change the length and/or height of the intervertebral spacer 310. For example, one embodiment of the support member 326 is an actuatable displacement element, as described in co-pending U.S. patent application Ser. No. ______, titled Active Vertebral Prosthetic Device, having the same filing date as the present application, and listing at least one common inventor (Attorney Docket No. P26217/31132.587), incorporated herein in its entirety by reference. Accordingly, the support member in some embodiments may be a piezoelectric actuator or an artificial muscle comprised of electroactive polymers (EAP) that actuates in response to electrical current. In other embodiments, the support member may be formed of ionic polymer-metal composites (IPMC) that actuate by voltage switching. In yet other embodiments, the support member is formed of a traveling wave actuator. In yet other embodiments, the support member may be hydraulically or pneumatically actuated. Still other embodiments include screws, ratchet means or other mechanical for changing the length and/or height. Electrical, thermal, and chemical actuators that change the length and/or height of the spacer 310 also are contemplated.

Actuation of the support member 326 causes deformation of the upper and lower beam members 318, 320, which affects rigidity and flexibility of the body 314. Accordingly, by actuating the support member 326, the properties of the non-rigid spacer 310 may be changed. For example, actuating the support member 326 increases or decreases the length of the body 314. If the body length decreases, the height increases. Likewise, if the body length increases, the height decreases.

The connecting element 316 extends between and connects the bodies 312, 314. Thus, the spacer 310 may provide relatively stable support to the adjacent vertebral bodies by connecting the bodies 312, 314 and increasing the size of the spacer footprint. The connecting element 316 may be formed of any material, either rigid or non-rigid, and in the embodiments shown, extends from the support members of each body 312, 314. In other embodiments, the connecting element 316 may extend from the beam members of one body to the beam members of the other body.

FIG. 13 shows a spacer 340 formed of bodies 342, 344 and a connecting element 346. The bodies 342, 344 may have any of the features of other embodiments described herein, and are shown with upper and lower beam members 348, 350 attached at ends 352, as well as with support members 354. The connecting element 346 includes a first and a second connector 356a-b that extend from the body 342 to the body 344. In the embodiment shown, the connectors 356a-b extend from the support members 354 and from the ends 352. However, it may extend from other parts of the bodies 342, 344. As described above, the connecting element provides stability to the spacer 340 by connecting bodies and increasing the size of the spacer footprint.

FIG. 14A and 14B show the spacer 340 in a compressed condition and an uncompressed condition respectively. The compressed condition shown in FIG. 14A may be a result of applied spinal loads applied at upper and lower outer surfaces 358, 360 of the upper and lower beam members 348, 350 or alternatively, may be the result of actuating the displacement element support member 354. Accordingly, the compressed condition may be the result of electrically actuating the support member 354. In the compressed condition shown in FIG. 14A, the spacer 340 has a first height H3 and a first length L3, while in the uncompressed condition, the spacer 340 has a second height H4 and a second length L4.

Although described with two bodies and one connecting element, the spacers may include additional bodies and connecting elements. For example, in some embodiments, the spacer includes three bodies placed so that their respective longitudinal axes form a rectangular shape. These bodies may or may not be attached to each other by connecting elements. In one exemplary embodiment, each of the three bodies is connected by connecting elements at ends to form the triangular shape. Other arrangements are contemplated.

The spacers may be implanted between the vertebrae 14, 16 using any common approach, including an anterior approach, a posterior approach, a posterior transforaminal approach, a far lateral approach, a direct lateral approach, among others. According to at least one of these approaches, an incision may be made in the patient to access the vertebrae and some or all of the affected disc and surrounding tissue may be removed. The superior endplate surface of the vertebra 14 may be milled, rasped, or otherwise resected to match the profile of the spacer to normalize stress distributions on the superior endplate surface of the vertebra 14 and/or to provide initial fixation prior to bone ingrowth. The preparation of the endplate of vertebra 14 may result in a flattened surface or in surface contours such as pockets, grooves, or other contours that may match corresponding features on the spacers. The inferior endplate of the vertebra 16 may be similarly prepared.

The spacer may then be introduced into the disc space. In some embodiments, the spacer is introduced through a cannula in a compressed condition, thereby minimizing the height of the spacer during insertion through the incision and during introduction to the disc space. Once in place, the spacer may be allowed or actuated to return to its uncompressed condition having a greater height, as limited by the adjacent bone structure. It should be noted that one or more spacers can be placed within the disc space. For example, some procedures may call for implanting a single spacer when using a lateral approach and two spacers when using a bilateral posterior approach. In any implantation, the spacers or their bodies may or may not be connected.

In some embodiments, having more than one body, such as in the embodiments disclosed in FIGS. 12 and 13, spacer components may be introduced one at a time, and assembled in place within the disc space. For example, a first body may be introduced and manipulated to a desired location, followed by introducing and connecting a connecting element to the first body, and then following with a second body that may be oriented and connected to the connecting element. Connecting the components in situ allows a less invasive surgical approach, as incisions may be kept relatively small. In other embodiments, the entire spacer is introduced as a single component.

When implanting spacers having an actuatable support member, the rigidity of the spacer may be controlled by actuating the support before or after implantation into the disc space. In some embodiments, the actuation may occur post-operatively while in other embodiments, the actuation occurs as a part of the surgical procedure. The actuation may be accomplished percutanteously using non-invasive procedures, such as RF wireless remote control systems. Alternatively, the actuation may be accomplished using wired remote control or alternatively, direct access during the surgical procedure, such as when hydraulically actuating with a syringe.

In some procedures, an operating physician may desire to fuse the spacer in place. In such circumstances, the physician may pack the spacer with bone growth promoting substances. For example, during the surgery, the hollow portion of a spacer body may be packed with bone graft material, tissue, or other osteogenic materials that promote bone growth. In other examples, the area between connectors of a connecting element, such as the connecting element 346 in FIG. 13, may be packed with bone graft material, tissue, or other osteogenic materials. In such procedures, the non-rigid spacer would become rigid over time, as the bone growth occurs. Osteogenic materials include, without limitation, autograft, allograft, xenograft, demineralized bone, synthetic and natural bone graft substitutes, such as bioceramics and polymers, and osteoinductive factors. A separate carrier to hold materials within the spacer can also be used. These carriers can include collagen-based carriers, bioceramic materials, such as BIOGLASS®, hydroxyapatite and calcium phosphate compositions. The carrier material may be provided in the form of a sponge, a block, folded sheet, putty, paste, graft material or other suitable form. The osteogenetic compositions may include an effective amount of a bone morphogenetic protein, transforming growth factor β1, insulin-like growth factor 1, platelet-derived growth factor, fibroblast growth factor, LIM mineralization protein (LMP), and combinations thereof or other therapeutic or infection resistant agents, separately or held within a suitable carrier material. It some embodiments, the body may include additional pores, apertures, or other features that provide communication through the beam members to promote bone growth at the bone-spacer interface.

Any of the features described with respect to one spacer embodiment may be used with any of the other spacer embodiments. For example and without limitation, the connecting member may be used with any of the spacer embodiments. In addition, although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” “right,” “cephalad,” “caudal,” “upper,” and “lower,” are for illustrative purposes only and can be varied within the scope of the disclosure. In the claims, means-plus-function clauses are intended to cover the elements described herein as performing the recited function and not only structural equivalents, but also equivalent elements.

We claim:

1. An intervertebral spacer, comprising:
a non-rigid body having an upper beam member and a lower beam member,
wherein the upper beam member includes a lower inner surface and includes an upper outer surface configured to interface with a vertebral plate of an upper vertebra,
wherein the lower beam member includes an upper inner surface and includes a lower outer surface configured to interface with a vertebral plate of a lower vertebra, and
wherein the upper inner surface of the lower beam member and the lower inner surface of the upper beam member define an oval-shaped hollow portion.

2. The intervertebral spacer of claim 1, wherein the body is deformable from a first height to a second height.

3. The intervertebral spacer of claim 2, wherein the body has a first length when the body is at the first height and a second length when the body is at the second height.

4. The intervertebral spacer of claim 1, wherein the upper and lower outer surfaces define an oval-shape.

5. The intervertebral spacer of claim 1, wherein the body has a width and a length, the length being at least 20% greater than the width.

6. The intervertebral spacer of claim 1, wherein the upper inner surface of the lower beam member and the lower inner surface of the upper beam member define an inner sidewall, the spacer including a support member extending from the inner sidewall across a portion of the hollow portion.

7. The intervertebral spacer of claim 6, wherein the support member is actuatable to affect at least one of a height and a width of the spacer.

8. The intervertebral spacer of claim 7, wherein the support member is one of mechanically actuated, electrically actuated, thermally actuated, and chemically actuated.

9. The intervertebral spacer of claim 1, wherein the upper and lower beam members are symmetrically disposed about a longitudinal axis.

10. The intervertebral spacer of claim 1, wherein the upper outer surface and the lower outer surface are substantially flat.

11. The intervertebral spacer of claim 10, wherein the upper outer surface and the lower outer surface include bone engaging features.

12. The intervertebral spacer of claim 1, wherein the upper outer surface and the lower outer surface are arc-shaped.

13. The intervertebral spacer of claim 12, wherein the upper outer surface and the lower outer surface are configured to slidably interface with the vertebral plates of the respective upper and lower vertebrae.

14. The intervertebral spacer of claim 12, wherein the upper outer surface and the lower outer surface include bone engaging features.

15. The intervertebral spacer of claim 14, wherein the bone engaging features are one of keels and a plurality of ridges.

16. The intervertebral spacer of claim 1, wherein the upper and lower beam members include a cut-out formed therein in a manner that affects the rigidity of the body.

17. The intervertebral spacer of claim 1, wherein the non-rigid body is a first non-rigid body, the intervertebral spacer comprising:
a second non-rigid body; and
a connecting member extending from the first non-rigid body to the second non-rigid body.

18. The intervertebral spacer of claim 17, wherein the connecting member is formed of a plurality of connectors.

19. An intervertebral spacer, comprising:
a non-rigid body having an upper beam member and a lower beam member, the upper beam member having an arcing upper outer surface configured to interface with a vertebral plate of an upper vertebra, and the lower beam member having an arcing lower outer surface configured to interface with a vertebral plate of a lower vertebra,
the body also having a hollow portion between the upper and lower outer surfaces,
the upper and lower beam members being connected in a manner such that the body can be compressed from a first height to a second smaller height.

20. The intervertebral spacer of claim 19, wherein the body has a first length when the body is at the first height and a second length when the body is at the second height.

21. The intervertebral spacer of claim 19, wherein the upper inner surface of the lower beam member and the lower inner surface of the upper beam member define an inner sidewall, the spacer including a support member extending from the inner sidewall across a portion of the hollow portion.

22. The intervertebral spacer of claim 21, wherein the support member is actuatable to affect at least one of a height and width of the spacer.

23. The intervertebral spacer of claim 19, wherein the upper outer surface and the lower outer surface are configured to slidably interface with the vertebral plates of the respective upper and lower vertebrae.

24. The intervertebral spacer of claim 19, wherein the upper outer surface and the lower outer surface include bone engaging features.

25. The intervertebral spacer of claim 19, wherein the upper and lower beam members include a cut-out formed therein in a manner that affects the rigidity of the body.

26. The intervertebral spacer of claim 19, wherein the non-rigid body is a first non-rigid body, the intervertebral spacer comprising:
a second non-rigid body; and
a connecting member extending from the first non-rigid body to the second non-rigid body.

27. The intervertebral spacer of claim 26, wherein the connecting member is formed of a plurality of connectors.

28. A method of surgically implanting an intervertebral spacer, comprising:
accessing an intervertebral space defined by an upper vertebra and a lower vertebra;
introducing a non-rigid intervertebral spacer having a body with an upper beam member and a lower beam member into the intervertebral space so that an arcing upper outer surface of the upper beam member interfaces with a vertebral plate of the upper vertebra, and so that an arcing lower outer surface of the lower beam member interfaces with a vertebral plate of the lower vertebra; and
compressing the body from a first height to a second smaller height.

29. The method of claim 28, wherein compressing the body includes changing the length of the body from a first length when the body is at the first height to a second length when the body is at the second height.

30. The method of claim 28, including supporting the body with a support member extending from an inner sidewall of the body.

31. The method of claim 30, wherein compressing the body includes actuating the support member.

32. The method of claim 28, wherein introducing the non-rigid vertebral spacer includes engaging the upper and lower vertebral plates with bone engaging features on the body.

33. The method of claim 28, further comprising:
introducing a second non-rigid intervertebral spacer having a second body with an upper beam member and a lower beam member into the intervertebral space; and
introducing a connecting member into the intervertebral space.

34. The method of claim 33, further comprising:
connecting the connecting member to the first and second non-rigid intervertebral spacers.

35. The method of claim 28, including introducing a bone growth promoting substance to the non-rigid intervertebral spacer.
